(19) United States
(12) Patent Application Publication
(10) Pub. No.: US 2017/0216577 A1
(43) Pub. Date: Aug. 3, 2017

(54) EQUIPMENT FOR APPLYING ACTIVE ELEMENTS TO THE SKULL OF A PATIENT

(71) Applicant: Jean-Tien, Mouaze (FR)

(72) Inventor: Jean Nguyen, Mouaze (FR)

(73) Assignee: Jean-Tien, Mouaze (FR)

(21) Appl. No.: 15/500,313

(22) PCT Filed: Jun. 17, 2015
(86) PCT No.: PCT/FR2015/051602

§ 371 Date: Jan. 30, 2017

(30) Foreign Application Priority Data
Jul. 30, 2014 (FR) 1457365

(51) Int. Cl. A61N 1/05; A61B 5/0476; A61N 5/06; A61B 5/00; A61N 1/04; A61N 1/00

(52) U.S. Cl. CPC A61N 1/0529; A61N 1/0456; A61N 1/00; A61N 5/0622; A61B 5/6814; A61B 5/0476; A61B 5/4064; A61N 2005/0647; A61N 2005/0651; A61N 2005/0663; A61N 2005/067

(57) ABSTRACT

Equipment for applying active elements to a cranium of a patient comprises a support and a plurality of active elements, wherein the support comprises a means of connection with a patient and means for positioning the active elements in order to excite N+M zones of the cranium, the N+M zones comprising the zones VG₁₉, VG₂₀, VG₂₁, C₄, C_{5R}, C_{5L}, C_{6R} and C_{6L}, N and M being two integer numbers, N being a number between 2 and 8, M being less than N/2.

### BACKGROUND

The present invention relates to the field of non-invasive stimulation of the brain and methods and equipment for applying treatments resulting from modern-medicine and energy-medicine knowledge.

It relates more precisely to treatment by actions performed on the cranium, and more particularly the physical equipment for implementing such treatments.

Various solutions for carrying out such treatments are known in the prior art.

By way of example, the American patent US 2014/0058189 describes a system and method for stimulating the brain for treating brain malfunctions. This patent describes a device for the non-invasive stimulation of the brain, using electromagnetic-field generators, the parameters of which are adjusted dynamically according to the electroencephalographic activity.

The American U.S. Pat. No. 6,537,304 describes apparatus for treating an ischaemic region of brain cells of the cranium, which comprises a cowl suitable for covering at least part of the cranium, at least one guidance device attached to the cowl, and a laser source serving to send a laser ray into the cranium by means of the guidance device. This guidance device may comprise an optical fibre or a waveguide.

The patent FR 2159851 describes an example of an electrode-holder helmet for the automatic positioning of EEG electrodes on the head of a patient.

The European patent EP 2489405 describes a portable device intended to produce phototherapy for the brain. It comprises a helmet covering part of the cranium of the patient. Light sources are positioned removably on this helmet.

The American patent application US 2010/204762 or the European patent EP 2533858 describe another example of a device for positioning brain phototherapy elements.

These patents relate to an apparatus and a method intended to indicate the locations of the sites for phototherapy treatment on the brain. In some embodiments, the apparatus is a helmet that can be worn by a patient. The helmet comprises a body designed to be worn on at least part of the scalp of the patient and a plurality of position indicators corresponding to a plurality of locations of treatment sites on the scalp of the patient where the light source is to be positioned sequentially so that the light coming from the light source is applied sequentially in order to irradiate at least part of the brain.

The American U.S. Pat. No. 8,644,940 describes a therapeutic brain stimulation system comprising at least two stimulation-signal emitters that produce stimulation signals from various positions in the direction of a common target region in the brain of a patient. Whereas the intensity of each stimulation signal is much too low to cause stimulation, the total stimulation signals cause stimulation and consequently have a therapeutic effect in the brain neurone cells of the target region. The stimulation signals accumulated in the target region can be adjusted so as not to negatively affect the anatomical structure of the brain neurone cells in the target region.

The patent application EP 2101640 relates to an electrode cap intended for electroencephalography that makes it possible to come into contact with the head of a person or of an animal without conductive gel between the scalp and the electrodes themselves. This electrode cap is intended to come into contact with the scalp of the head in electroencephalography with a certain number of electrodes in the form of a pin intended to come into contact with the scalp, and an electrode-holding member characterised in that the electrodes are mounted on this electrode-holding member via at least one elastic articulation.

The patent EP 1663392 relates to treatment apparatus intended to treat the brain of a patient. This treatment apparatus comprises a light generator having an output emission zone disposed so as to irradiate part of the brain with effective power density and light wavelength. Said treatment apparatus further comprises an element interposed between the light generator and the scalp of the patient.

The patent EP 1429709 relates to a device for applying at least one laser needle to the body of a patient. Said device comprises a fixing element able to be adapted to a part of the body of the patient. Said fixing element has at least one guide able to receive the laser needle so that the latter can be moved with respect to the part of the body by means of said guide.

The patent application EP 2496135 relates to a device for positioning electrodes on the scalp of a user. The device comprises a casing, for example a listening-device helmet that can be placed on the head of a user. An elastic element and a plurality of electrodes are positioned so that, once the housing is in place on the head of a user, the elastic element at least partially follows the curvature of the head of the user. The force of the elastic element due to the stretching that it undergoes when said housing is placed on the head of the user means that the elastic element exerts a pressure on at least some of the plurality of electrodes, in the direction of the scalp.

Advantageously, the active elements are sensors for measuring the brain activity or a combination of sensors and transmitters for measuring the brain activity and stimulating the brain simultaneously.

The solutions in the prior art result in erratic positionings of the active elements, and empirical and poorly controlled treatments.

### SUMMARY

The present invention aims to remedy these drawbacks by proposing equipment for applying active elements to the cranium of a patient, comprising a support and a plurality of active elements, characterised in that said support comprises a means of connection with a patient and means for positioning said active elements in order to excite N+M zones of the cranium, said N zones comprising the zones [VG₁₉, VG₂₀, V₂₁, C₄, C_{5R}, C_{5L}, C_{6R} and C_{6L}], N and M being two integer numbers, N being a number between 2 and 8, M being less than N/2.

Advantageously N is a number between 4 and 8 and M is equal to 0.

Preferably, said active elements are low-power lasers. “Low power” within the meaning of the present patent will mean an electrical power of less than 60 mW.

Advantageously, said laser has a power of between 1 and 15 mW.

Preferably, each of the active elements consists of a laser emitting a red beam with a wavelength of approximately 650 nm.

According to a variant, said active elements consist of light emitting diodes (LEDs).

According to a variant, said active elements consist of electromagnetic-field generators.

Preferably, the equipment according to the invention comprises a means for adjusting the energy applied to each of said active elements.

### BRIEF DESCRIPTION OF THE DRAWINGS

The present invention will be understood better from a reading of the following description, referring to a non-limitative example embodiment, where:

FIG. 1 shows a schematic view of equipment according to the invention

FIG. 2 shows a schematic front view of the location of the zones where the active elements are positioned.

### DETAILED DESCRIPTION

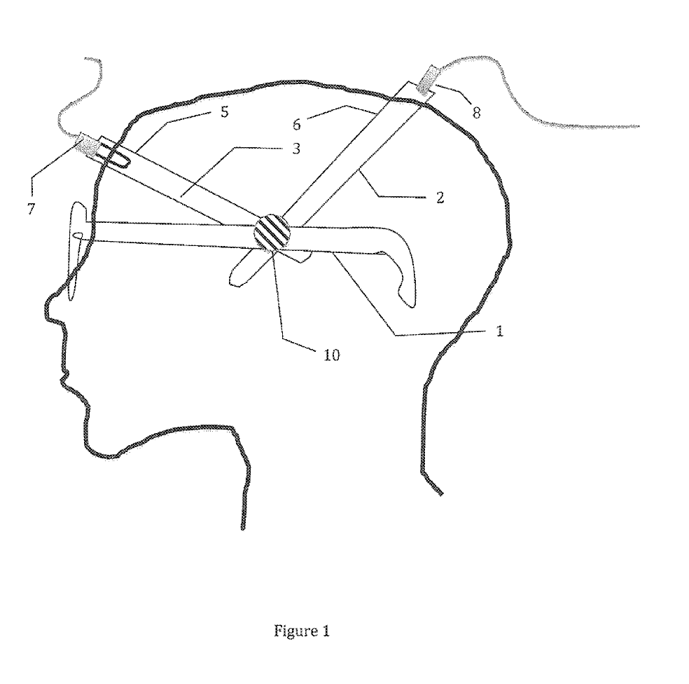

FIG. 1 shows a schematic view of equipment according to the invention.

It consists of a mount, having a central or frontal support for the cranium with lateral extensions (1). This mount constitutes a reference frame for positioning the sensors facing certain zones of the cranium.

The lateral extensions of the mount (1) support, at the temporal zone of the cranium, a plurality of arches (2, 3) in the form of a head grip, articulated on each lateral extension (1) by means of a pivot (10) allowing angular adjustment with respect to the cranial reference frame defined by the mount, and then the locking of the relative orientations of the arches (2, 3).

These arches (2, 3) have apertures (5, 6) allowing the movement, on paths defined by the arches (2, 3), of active elements (7, 8). These active elements are for example laser diodes emitting in the red range (650 nanometres) with a power of 10 mW. They are supplied by an electronic circuit delivering for each of the diodes a modulated signal with a peak voltage of 3 volts.

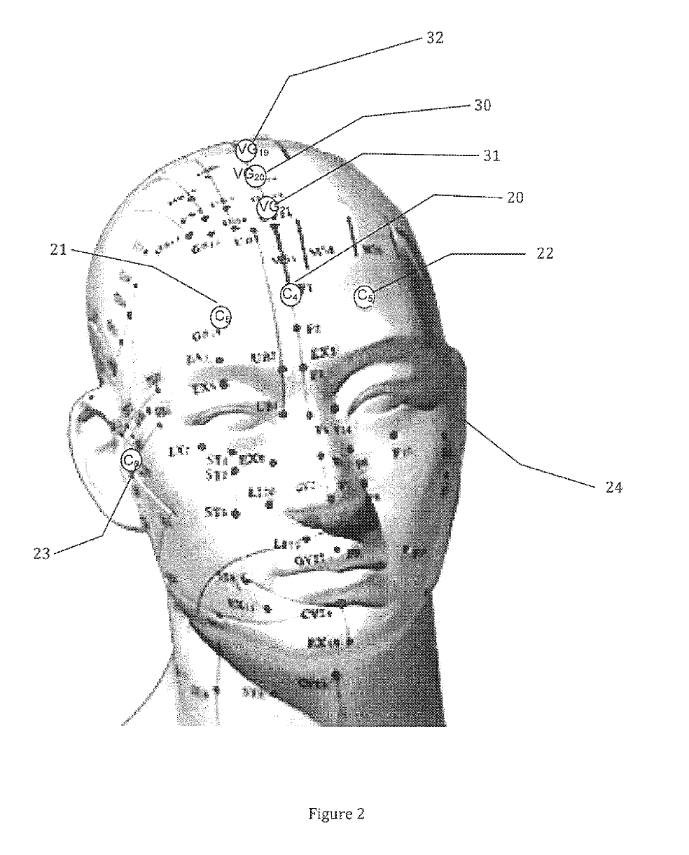

FIG. 2 shows a schematic view of the action zones of the active elements positioned by a device, for example the one described in FIG. 1.

These action zones are chosen mainly from the following zones:
A) The zones (30 to 32) C₁, C₂ and C₃ situated on the meridian of the governing vessel.

They comprise the 20^{th} point of the governing/Ren Mai vessel (VG₂₀). This VG₂₀ zone (30) is situated at approximately five centimetres to the rear of the forehead hairline and at approximately seven centimetres above the nape hairline, at the intersection of the median line of the cranium and a line connecting the end of the two ears (the apex), on the epicranial aponeurosis and between the two parietal foramina.

The point VG₁₉ (or Chakra 7) (32) is situated at the crown of the head. The point VG₂₁ (31) is situated on the median line, symmetrically opposite point (32) with respect to point (30).
B) The four zones C₄, C₅ and C₆ belong to the points that are not on meridians of the face.

The point C₄ (20) is situated at the middle of the forehead. The two points C₅ (21, 22) are situated on either side of the point C₄ (20), on the horizontal line passing through this point C₄, vertically above the middle of each eyebrow.

The two points C₆ (23, 24) are situated on the meridians of the triple burner, at the temple.

The apparatus is used by positioning four to eight actuators on the support, to correspond to points mainly chosen from the aforementioned eight points. The choice of the aforementioned points is defined by the medical practitioner for precise therapeutic treatment.

When the patient is equipped, each of the diodes is supplied at a frequency of between 4 and 10 Hz, for a period of 10 to 30 minutes.

The equipment can be produced in numerous alternatives. In particular, it can take the form of a support pillow for the head and neck comprising a structure support element, forming a support for the nape of the neck. The active elements are fixed to the structure support element, as well as to extensions supporting the active elements for the zones that are not in contact with the surface of the pillow.

1. Equipment for applying active elements to a cranium of a patient, comprising:
a support and a plurality of active elements, wherein said support comprises a means of connection with a patient and means for positioning said active elements in order to excite N+M zones of the cranium, said N+M zones comprising the zones VG₁₉, VG₂₀, VG₂₁, C₄, C_{5R}, C_{5L}, C_{6R} and C_{6L}, N and M being two integer numbers, N being a number between 2 and 8, M being less than N/2.

2. The equipment for applying active elements to the cranium of a patient according to claim 1, wherein N is a number between 4 and 8 and M is equal to 0.

3. The equipment for applying active elements to the cranium of a patient according to claim 1, wherein said active elements are laser diodes with a power of less than 60 mW.

4. The equipment for applying active elements to the cranium of a patient according to claim 3, wherein said laser has a power of between 1 and 15 mW.

5. The equipment for applying active elements to the cranium of a patient according to claim 3, wherein said laser is a laser emitting in the red range.

6. The equipment for applying active elements to the cranium of a patient according to claim 1, wherein said active elements consist of electromagnetic-field generators.

7. The equipment for applying active elements to the cranium of a patient according to claim 1, wherein the equipment comprises a means for adjusting the energy applied to each of said active elements.

8. The equipment for applying active elements to the cranium of a patient according to claim 7, further comprising:
a central or frontal support for the cranium with lateral extensions that constitute a reference frame for positioning active elements facing precise zones of the cranium;
lateral extensions that support, at the temporal zone of the cranium, a plurality of arches articulated on each lateral extension by means of a pivot allowing angular adjustment with respect to the cranial reference frame defined by the mount, and then the locking of the relative orientations of the arches; and
arches that have apertures allowing the movement of the active elements on the paths defined by the arches.

9. The equipment according to claim 8, wherein the active elements are sensors configured to measure brain activity.

10. The equipment according to claim 8, wherein the active elements are transmitters or energy sources for stimulating the brain.

11. The equipment according to claim 8, wherein the active elements are a combination of sensors and transmitters configured to measure the brain activity and to stimulate the brain simultaneously.
